SEQUENCE LISTING (18 sequences)

SEQ ID NO: 1 - DNA, 24 bp, Artificial Sequence
  forward primer 1
ctatagtagaagaaccgtatttac

SEQ ID NO: 2 - DNA, 24 bp, Artificial Sequence
  forward primer 2
gattatctcagctattttaatgtc

SEQ ID NO: 3 - DNA, 21 bp, Artificial Sequence
  forward primer 3
tttaggacaacaaagattggg

SEQ ID NO: 4 - DNA, 20 bp, Artificial Sequence
  forward primer 4
cttcgtgatttgcgtgatgc

SEQ ID NO: 5 - DNA, 21 bp, Artificial Sequence
  forward primer 5
tttaggacaacaaagattggg

SEQ ID NO: 6 - DNA, 22 bp, Artificial Sequence
  forward primer 6
cttacatgactatgaaaatccg

SEQ ID NO: 7 - DNA, 21 bp, Artificial Sequence
  forward primer 7
tttaggacaacaaagattggg

SEQ ID NO: 8 - DNA, 22 bp, Artificial Sequence
  forward primer 8
gcaattaaagcgccagttgctg

SEQ ID NO: 9 - DNA, 22 bp, Artificial Sequence
  reverse primer 1
cagcaactggcgctttaattgc

SEQ ID NO: 10 - DNA, 22 bp, Artificial Sequence
  reverse primer 2
cggattttcatagtcatgtaag

SEQ ID NO: 11 - DNA, 20 bp, Artificial Sequence
  reverse primer 3
gcatcacgcaaatcacgaag

SEQ ID NO: 12 - DNA, 23 bp, Artificial Sequence
  reverse primer 4
gtcttattaaaggaagcaattgc

SEQ ID NO: 13 - DNA, 24 bp, Artificial Sequence
  reverse primer 5
gacattaaaatagctgagataatc

SEQ ID NO: 14 - DNA, 23 bp, Artificial Sequence
  reverse primer 6
gtcttattaaaggaagcaattgc

SEQ ID NO: 15 - DNA, 24 bp, Artificial Sequence
  reverse primer 7
gtaaatacggttcttctactatag

SEQ ID NO: 16 - DNA, 23 bp, Artificial Sequence
  reverse primer 8
gtcttattaaaggaagcaattgc

SEQ ID NO: 17 - DNA, 27 bp, Artificial Sequence
  oligonucleotide used for PCR amplification in example 1
gcgaagcttcaaataggggttccgcgc

SEQ ID NO: 18 - DNA, 28 bp, Artificial Sequence
  oligonucleotide used for PCR amplification in example 1
gcgactagtgggaaaactgtccataccc

(19) United States
(12) Patent Application Publication
(10) Pub. No.: US 2008/0253990 A1
(43) Pub. Date: Oct. 16, 2008

(54) Self-Containing Lactobacillus Strain

(75) Inventors: Lothar Steidler, Drongen (BE); Sabine Neirynck, Drongen (BE)

Correspondence Address: TRASK BRITT, P.O. BOX 2550, SALT LAKE CITY, UT, 84110, US

(73) Assignee: UNIVERSITY COLLAGE COOK, CORK (IE)

(21) Appl. No.: 11/596,715

(22) PCT Filed: May 18, 2005
(86) PCT No.: PCT/EP05/52296

§ 371 Date: Mar. 7, 2008

(30) Foreign Application Priority Data
May 18, 2004 (EP) 04102202.1

(51) Int. Cl. A61K 47/00; C12N 1/21; A61K 38/20; A61P 1/00

(52) U.S. Cl. 424/85.2; 435/252.3; 514/772

(57) ABSTRACT

The invention relates to a recombinant Lactobacillus strain, with limited growth and viability in the environment. More particularly, it relates to a recombinant Lactobacillus that can only survive in a medium where thymidine is present. By this strict dependency upon thymidine, thymidineless death is rapidly induced in this recombinant strain. A preferred embodiment is a Lactobacillus that may only survive in a host organism where thymidine is present, but cannot survive outside the host organism in absence of this medium compound. Moreover, the Lactobacillus strain can be transformed with prophylactic and/or therapeutic molecules and can, as such, be used to treat diseases such as, but not limited to, inflammatory bowel diseases.

### CROSS-REFERENCE TO RELATED APPLICATIONS

This is a national phase entry under 35 U.S.C. §371 of International Patent Application PCT/EP2005/052296, filed May 18, 2005, published in English as International Patent Publication WO 2005/111194 A1 on Nov. 24, 2005, which claims the benefit under 35 U.S.C. §119 of European Patent application 04102202.1, filed May 18, 2004.

### FIELD OF THE INVENTION

The invention relates to a recombinant Lactobacillus strain with limited growth and viability in the environment. More particularly, it relates to a recombinant Lactobacillus that can only survive in a medium where thymidine is present. By this strict dependency upon thymidine, thymidineless death is rapidly induced in this recombinant strain. A preferred embodiment is a Lactobacillus that may only survive in a host organism where thymidine is present, but cannot survive outside the host organism in absence of this medium compound. Moreover, the Lactobacillus strain can be transformed with prophylactic and/or therapeutic molecules and can, as such, be used to treat diseases such as, but not limited to, inflammatory bowel diseases.

### BACKGROUND OF THE INVENTION

Lactic acid bacteria have long time been used in a wide variety of industrial fermentation processes. They have “generally regarded as safe” status, making them potentially useful organisms for the production of commercially important proteins. Indeed, several heterologous proteins, such as Interleukin-2, have been successfully produced in Lactococcus spp (Steidler et al., 1995). It is, however, unwanted that such genetically modified microorganisms are surviving and spreading in the environment.

To avoid unintentional release of genetically modified microorganisms, special guidelines for safe handling and technical requirements for physical containment are used. Although this may be useful in industrial fermentations, the physical containment is generally not considered as sufficient, and additional biological containment measures are taken to reduce the possibility of survival of the genetically modified microorganism in the environment. Biological containment is extremely important in cases where physical containment is difficult or even not applicable. This is, amongst others, the case in applications where genetically modified microorganisms are used as live vaccines or as vehicle for delivery of therapeutic compounds. Such applications have been described, e.g., in WO 97/14806, which discloses the delivery of biologically active peptides, such as cytokines, to a subject by recombinant non-invasive or non-pathogenic bacteria. WO 96/11277 describes the delivery of therapeutic compounds to an animal, including humans, by administration of a recombinant bacterium encoding the therapeutic protein. Steidler et al. (2000) describe the treatment of colitis by administration of a recombinant Lactococcus lactis secreting interleukin-10. Such a delivery may indeed be extremely useful to treat a disease in an affected human or animal, but the recombinant bacterium may act as a harmful and pathogenic microorganism when it enters a non-affected subject, and an efficient biological containment that avoids such unintentional spreading of the microorganism is needed.

Although a sufficient treatment can be obtained using Lactococcus, it has as the main disadvantages that the bacterium is not colonizing and that the medication should applied in a continuous way to ensure the effect. A colonizing strain like Lactobacillus would have the advantage that a similar effect can be used with a single dose or a limited number of doses. However, similar to the Lactococcus case, a stringent biological containment system is needed to avoid the dissemination of the bacterium in the environment.

Biological containment systems for host organisms may be passive, based on a strict requirement of the host for specific growth factor or a nutrient that is not present or present in low concentrations in the outside environment, or active, based on so-called suicidal genetic elements in the host, whereby the host is killed in the outside environment by a cell-killing function, encoded by a gene that is under control of a promoter only being expressed under specific environmental conditions.

Passive biological containment systems are well known in microorganisms such as Escherichia coli or Saccharomyces cerevisiae. Such E. coli strains are disclosed, e.g., in U.S. Pat. No. 4,100,495. WO 95/10621 discloses lactic acid bacterial suppressor mutants and their use as means of containment in lactic acid bacteria, but in that case, the containment is on the level of the plasmid, rather than on the level of the host strain, and it stabilizes the plasmid in the host strain, but doesn't provide containment for the genetically modified host strain itself.

Active suicidal systems have been described by several authors. Such systems consist of two elements: a lethal gene and a control sequence that switches on the expression of the lethal gene under non-permissive conditions. WO 95/10614 discloses the use of a cytoplasmatically active truncated and/or mutated Staphylococcus aureus nuclease as lethal gene. WO 96/40947 discloses a recombinant bacterial system with environmentally limited viability, based on the expression of either an essential gene, expressed when the cell is in the permissive environment, and is not expressed or temporarily expressed when the cell is in the non-permissive environment and/or a lethal gene, wherein expression of the gene is lethal to the cell and the lethal gene is expressed when the cell is in the non-permissive environment but not when the cell is in the permissive environment. WO 99/58652 describes a biological containment system based on the relE cytotoxin. However, most systems have been elaborated for Escherichia coli (Tedin et al., 1995; Knudsen et al., 1995; Schweder et al., 1995) or for Pseudomonas (Kaplan et al., 1999; Molina et al., 1998).

An interesting alternative is to use a mutation in the gene for thymidylate synthase as containment system. Both prokaryotic and eukaryotic cells carrying such mutation are unable to grow on low concentration of thymidine or thymine, and undergo cell death in response to this starvation. This phenomenon is known as thymineless death (Goulian et al., 1986; Ahmad et al., 1998). A containment system based on this mutation has been described for Lactobacillus acidophilus by Fu and Xu (2000), using the thyA gene from Lactobacillus casei as selective marker. The thyA mutant used has been selected by spontaneous mutagenesis and trimethoprim selection. Such a mutation is prone to reversion and the thyA gene of another Lactobacillus species is used to avoid the reversion of the mutation by inrecombination of the marker gene. Indeed, reversion of the thyA mutation is a problem, and especially in absence of thymine or thymidine in the medium, the mutation will revert at high frequency, whereby the strain is losing its containment characteristics. For an acceptable biological containment, a non-reverting mutant is wanted.

Non-reverting mutants can be obtained by gene disruption. A containment system based on this disruption has been described for Lactococcus (Steidler et al., 2003). However, although the thyA gene of Lactobacillus casei has been cloned and mutated by site-directed mutagenesis, it was only tested in E. coli, and never used for gene replacement in a Lactobacillus strain. Although transformation techniques for Lactobacillus are known to the person skilled in the art, gene disruption of thyA in Lactobacillus has never succeeded and is clearly not evident.

Surprisingly, we were able to construct the thyA disruption in Lactobacillus. Even more surprisingly, we found that survival of this disruption mutant is strictly thymidine-dependent, and that the mutant cannot be rescued by addition of thymine to the medium. The latter is especially surprising, as it is generally accepted that thyA mutants can be rescued either by addition of thymidine or thymine to the medium (Fu and Xu, 2000; Ahmad et al. 1998). The viability of such a strain is rapidly decreasing in absence of thymidine (even in presence of thymine) and, therefore, it is an ideal host strain when biological containment is needed. Both the rapid induction of thymidineless death, which is faster than for the previously described Lactococcus strain, and the fact that the strain cannot be rescued by thymine, makes it an ideal strain for delivery of prophylactic and/or therapeutic molecules into a living animal, including humans.

### SUMMARY OF THE INVENTION

It is the objective of the present invention to provide a suitable biological containment system for Lactobacillus.

A first aspect of the invention is an isolated strain of Lactobacillus sp. comprising a defective recombinant thymidylate synthase gene (thyA), whereby survival of the strain is strictly dependent upon the presence of thymidine. Preferably, the defective recombinant gene is situated in the chromosome and inactivated by gene disruption. “Gene disruption,” as used herein, includes disruption by insertion of a DNA fragment, disruption by deletion of the gene, or a part thereof, as well as exchange of the gene or a part thereof by another DNA fragment, and the disruption is induced by recombinant DNA techniques, and not by spontaneous mutation. Preferably, disruption is the exchange of the gene, or a part thereof, by another functional gene. Preferably, the defective recombinant thymidylate synthase gene is a non-reverting mutant gene.

A “non-reverting mutant,” as used herein, means that the reversion frequency is lower than 10⁻⁸, preferably the reversion frequency is lower than 10⁻¹⁰, even more preferably, the reversion frequency is lower than 10⁻¹², even more preferably, the reversion frequency is lower than 10⁻¹⁴, most preferably, the reversion frequency is not detectable using the routine methods known to the person skilled in the art. Preferably, Lactobacillus sp. is Lactobacillus salivarius. Even more preferably, Lactobacillus is Lactobacillus salivarius subsp. salivarius strain UCC118. A non-reverting thyA mutant strain can be considered as a form of active containment, as it will undergo cell death in response to thymidine starvation (Ahmad et al., 1998).

Contrary to all thyA mutants previously described, the mutant is unable to be rescued by thymine, and will undergo cell death even if thymine is present in the medium. To be “rescued,” as used herein, means that the strain cannot grow upon addition of a certain concentration of thymine to a medium where all necessary compounds for growth of the strain are present, except thymidine. Preferably, the mutant will undergo thymidineless death even in presence of thymine at a concentration of 25 μg/ml, more preferably 30 μg/ml, more preferably 40 μg/ml, even more preferably 50 μg/ml, most preferably 100 μg/ml. The mutant is further characterized by a rapid decrease of viability in absence of thymidine in the medium. Preferably, the initial decrease in viability in absence of thymidine is as fast as 2 log units colony forming units (cfu) in 16 hours, even more preferably the initial decrease is 2 log units cfu in 12 hours, most preferably the initial decrease is as fast as 2 log units cfu in 8 hours. The initial decrease in viability is measured as cfu after time X (here, 16, 12 or 8 hours, respectively), compared with the colony forming units at time 0, when the strain is kept at 37° C. in MRS medium devoid of thymidine.

Previously described Lactobacillus thyA mutants, similar to other thyA mutants, could always be rescued by addition of thymine or thymidine to the medium. However, especially in cases where the concentration of thymine and/or thymidine cannot be carefully controlled, a strict dependence upon thymidine in the medium is a strong advantage for biological containment. As a non-limiting example, this may be the case in industrial fermentations using bulk media that may be contaminated with traces of thymine. Furthermore, the present invention discloses that such a strain is especially useful in these cases where the strain is used as a delivery vehicle in an animal body, including the human body. When such a transformed strain is given, for example, orally to an animal, including humans, it survives in the gut and produces homologous and/or heterologous proteins, such as, but not limited to, human interleukin-10, that may be beneficial for that animal. The fact that the mutant cannot be rescued by thymine provides a better containment, especially when used in the human and animal body, where the residual concentration of thymidine or thymine in the feces cannot be controlled.

Therefore, another aspect of the invention is the use of a Lactobacillus strain according to the invention as a biologically contained strain for the delivery of prophylactic and/or therapeutic molecules. Preferably, delivery requires a biological containment under conditions whereby the thymidine and/or thymine concentration cannot be strictly controlled, such as, but not limited to, the delivery of the prophylactic and/or therapeutic molecules in animals, including humans, to prevent and/or treat diseases. “Conditions whereby the thymidine and/or thymine concentration cannot be strictly controlled,” as used herein, means that there is no direct control on the concentration, such as control of the concentration by an active and controlled addition or removal of thymine or thymidine. Preferably, the thymine- or thymidineless conditions are generated by natural processes, such as exhaustion of thymidine by uptake of thymidine in the intestine. The delivery of prophylactic and/or therapeutic molecules has been disclosed, as a non-limiting example, in WO 97/14806 and in WO 98/31786. Prophylactic and/or therapeutic molecules include, but are not limited to, polypeptides such as insulin, growth hormone, prolactine, calcitonin, group 1 cytokines, group 2 cytokines, group 3 cytokines, neuropeptides and antibodies, and polysaccharides, such as polysaccharide antigens from pathogenic bacteria.

In a preferred embodiment, the thyA gene of a Lactobacillus sp. strain, preferably Lactobacillus salivarius, is disrupted and replaced by a functional human interleukin-10 expression cassette and the strain can be used for delivery of IL-10. The interleukin-10 expression unit is preferably, but not limited to, a human interleukin-10 expression unit or gene encoding for human interleukin-10. Therefore, a preferred embodiment is the use of a Lactobacillus sp. strain according to the invention to deliver human interleukin-10. Methods to deliver the molecules and methods to treat diseases such as inflammatory bowel diseases are explained in detail in WO 97/14806 and WO 00/23471 to Steidler et al. and in Steidler et al. (2000), that are hereby incorporated by reference. The present invention demonstrates that the strain according to the invention surprisingly passes the gut at the same speed as the control strains and shows that their loss of viability is indeed not different from that of the control strains. However, once the strain is secreted in the environment, e.g., in the feces, it is not able to survive any longer. The fact that the deletion mutant can survive in the intestine, and more specifically in the ileum, and as such can be used as a biologically contained delivery strain, is especially surprising, as it is solely dependent upon thymidine.

Another aspect of the invention is a pharmaceutical composition comprising a Lactobacillus sp. thyA disruption mutant, according to the invention. As a non-limiting example, the bacteria may be encapsulated to improve the delivery to the intestine. Methods for encapsulation are known to the person skilled in the art and are disclosed, amongst others, in EP0450176.

Still another aspect of the invention is the use of a strain according to the invention for the preparation of a medicament. Preferably, the medicament is used to treat Crohn's disease or inflammatory bowel disease.

### BRIEF DESCRIPTION OF THE FIGURES

FIG. 1: All Lactobacillus salivarius strains are indicated by their strain codes (UCC118, TGB078, TGB092) where relevant.

Panel A: Schematic overview of gene exchange between UCC118 thyA (hatched) and hIL-10 (black). Target DNA for homologous recombination (gray), 1 Kb in size, is residing both on the chromosome of UCC118, as well as on a non-replicative, erythromycin-(Em-) resistance marker-positive plasmid, both upstream and downstream of thyA and hIL-10, respectively. UCC118 chromosomal DNA (thick black line) flanks both upstream and downstream target DNA. After introduction of the non-replicative plasmid in UCC118 (1), the transformation mixture is incubated in the presence of Em. This allows for the selection of homologous recombination events at either the upstream or downstream target (2), which can be discriminated by PCR using 1F/1R or 2F/2R oligonucleotides. Repeated growth in the absence of Em and in the presence of 50 μg/ml thymidine allows for a second recombination to occur (3), which can be detected by combined 1F/1R and 2F/2R PCR. Em negative, 1F/1R 2F/2R PCR-positive clones have the desired genetic structure (4).

Panel B: Detail of parent strain Lactobacillus salivarius UCC118 and resulting strains Lactobacillus salivarius TGB078 and Lactobacillus salivarius TGB092. TGB092 carries the Lactococcus lactis thyA promoter (PthyA, GenBank AF462070).

FIG. 2: PCR identification of gene exchange between Lactobacillus salivarius UCC118 thyA (hatched) and hIL-10 (black), resulting in strains Lactobacillus salivarius TGB078 and Lactobacillus salivarius TGB092. All strains are indicated by their strain codes (UCC118, TGB078, TGB092). Panel A shows a schematic overview of the different PCR reactions. Panel B shows agarose gel electrophoresis data of the relevant molecular size interval. Numbers 1 through 8 indicate the different PCR reactions in both panels.

PCR1: detection of thyA in UCC118, not in TGB078 and TGB092.

PCR2: detection of hIL10 in TGB078 and TGB092, not in UCC118.

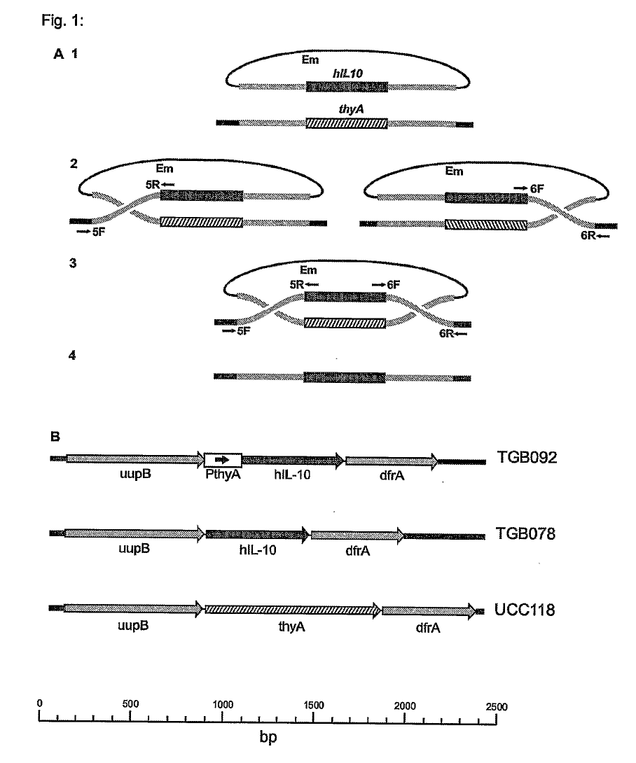

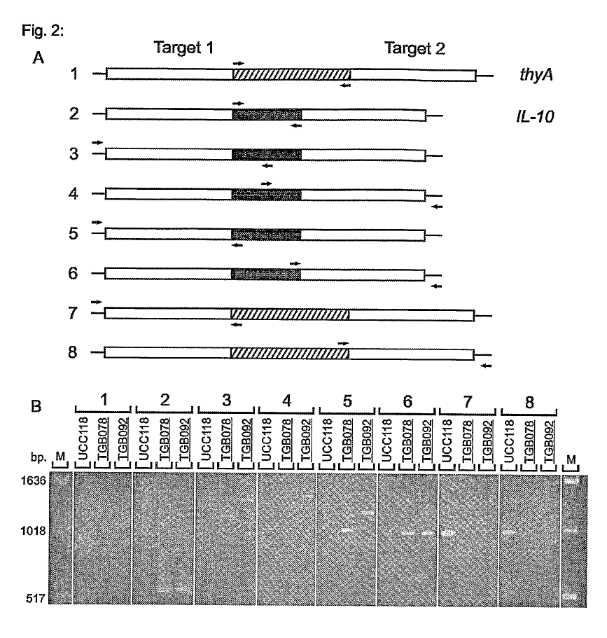

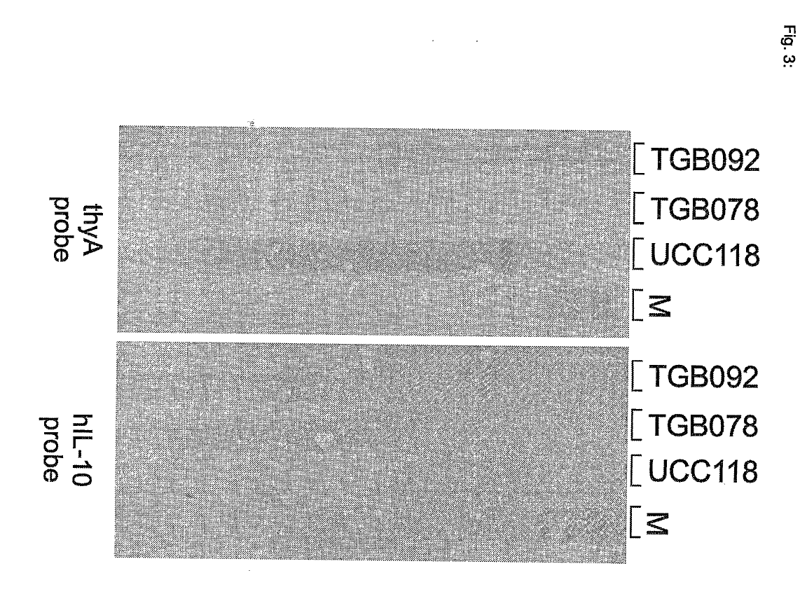

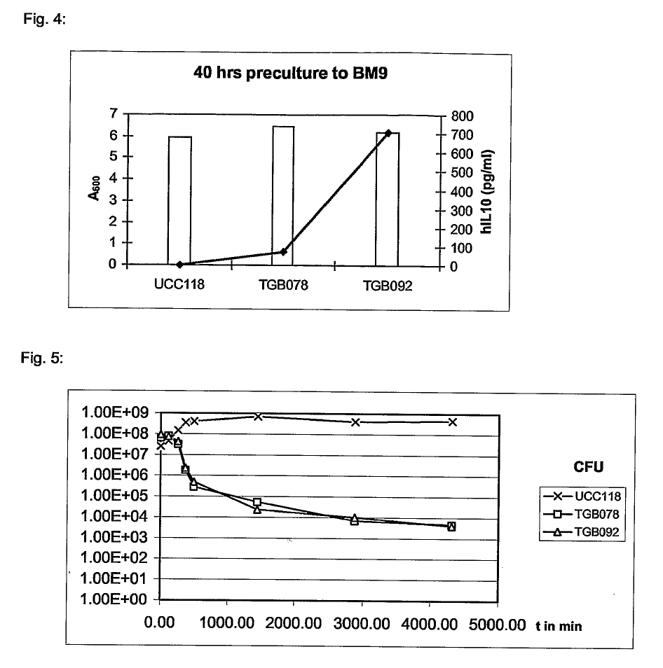

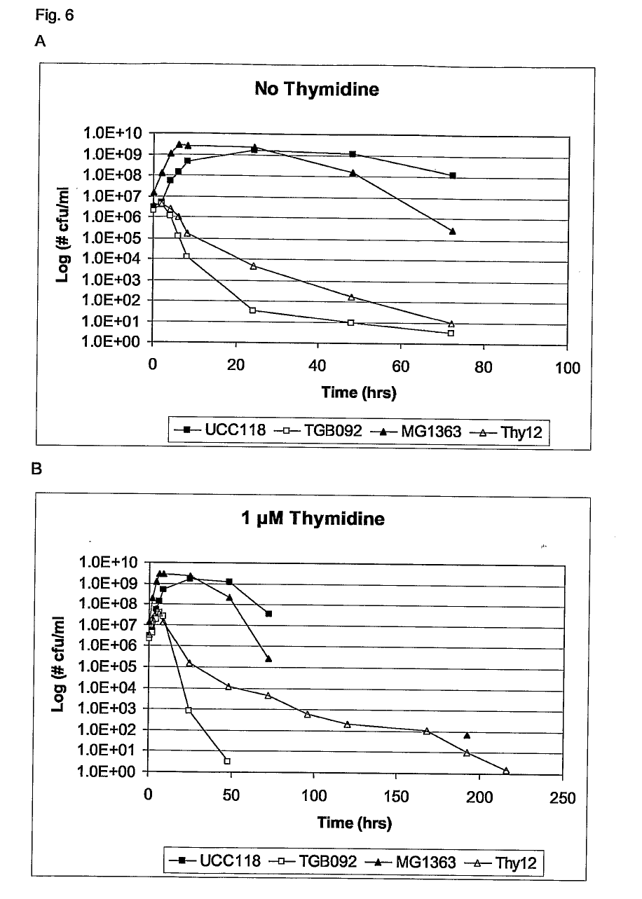

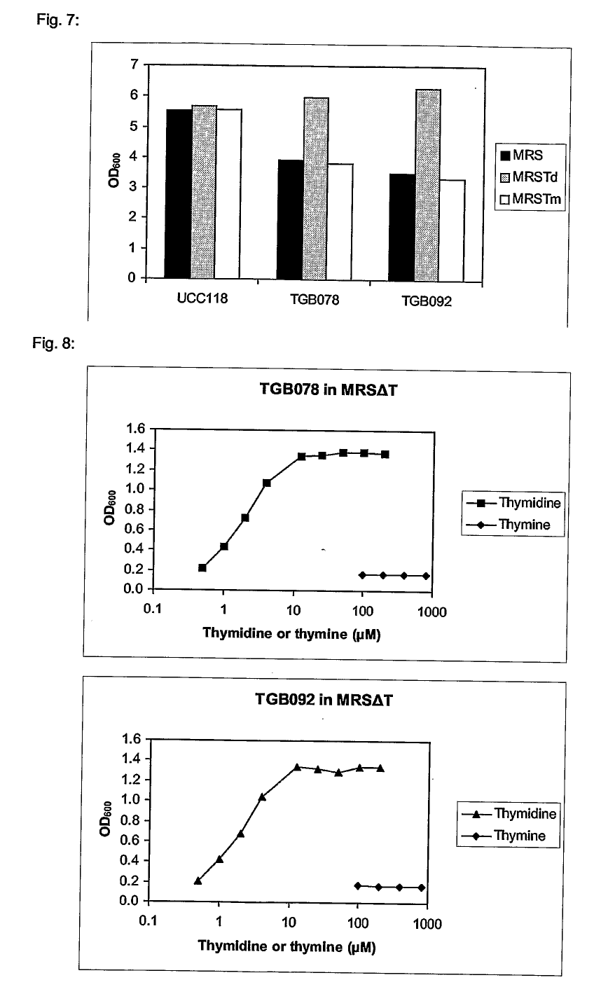

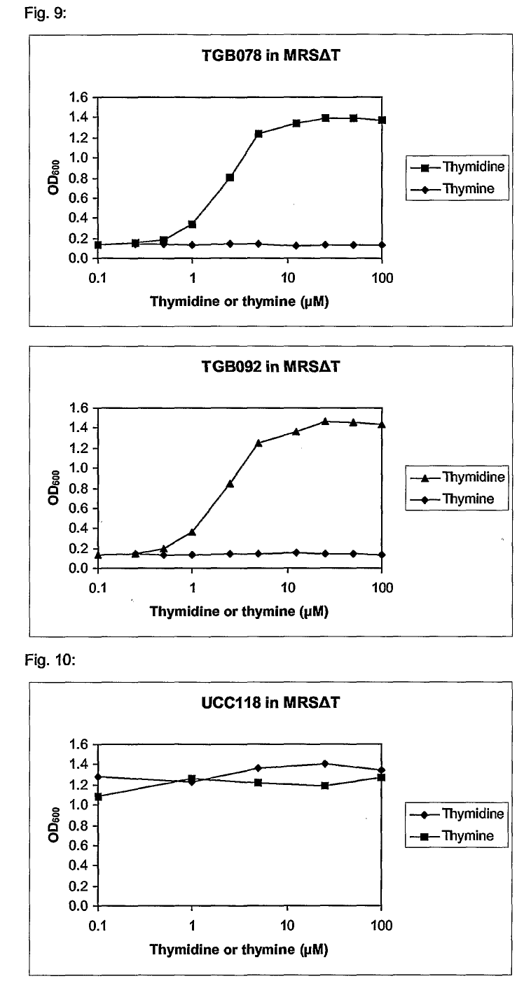

PCR3: detection of hIL10 attached to upstream genomic DNA outside the target region in TGB078 and TGB092, not in UCC118. Size differences are a result of differences in the hIL-10 promoter regions, as detailed in FIG. 1, Panel B.

PCR4: detection of hIL-10 attached to downstream genomic DNA outside the target region in TGB078 and TGB092, not in UCC118.

PCR5: detection of hIL-10 attached to upstream genomic DNA outside the target region in TGB078 and TGB092, not in UCC118. Size differences are a result of differences in the hIL-10 promoter regions, as detailed in FIG. 1, Panel B.

PCR6: detection of hIL-10 attached to downstream genomic DNA outside the target region in TGB078 and TGB092, not in UCC118.

PCR7: detection of thyA attached to upstream genomic DNA outside the target region in UCC118, not in TGB078 and TGB092.

PCR8: detection of thyA attached to downstream genomic DNA outside the target region in UCC118, not in TGB078 and TGB092.

FIG. 3: Southern blot hybridization of Lactobacillus salivarius UCC118, Lactobacillus salivarius TGB078 and Lactobacillus salivarius TGB092. All strains are indicated by their strain codes (UCC118, TGB078, TGB092). Complete chromosomal DNA was prepared with a Qiagen Dneasy tissue kit, as described by the manufacturer, with the adaptation that the bacterial cell wall was digested with lysozyme during the first step of the protocol. The DNA preparations were cut with EcoR1 and separated on a 1.2% agarose gel, alongside with Roche DIG-labeled DNA molecular weight marker VII. The DNA was transferred to a nylon membrane and revealed with DIG-labeled thyA and hIL-10 probes. All DIG labeling and detection was performed as described by the manufacturer (Roche). UCC118 shows a signal of the appropriate size with the thyA probe and not with the hIL-10 probe. TGB078 and TGB092 show no signal with the thyA probe but show signals of appropriate sizes with the hIL-10 probe. Size differences of the latter originate from the differences in promoter structure of both TGB078 and TGB092, as was outlined in FIG. 1.

FIG. 4: IL-10 production by Lactobacillus salivarius UCC118, Lactobacillus salivarius TGB078 and Lactobacillus salivarius TGB092. All strains are indicated by their strain codes (UCC118, TGB078, TGB092). Single colonies of all strains were inoculated in MRS supplemented with 50 μg/ml of thymidine and incubated for 40 hours at 37° C. Bacteria were harvested by centrifugation, resuspended in BM9 (buffered M9 growth medium) supplemented with 50 μg/ml of thymidine, and incubated for five hours at 37° C. IL-10 in the culture supernatant was determined by ELISA (Becton Dickinson).

FIG. 5: Survival in the absence of thymidine of Lactobacillus salivarius UCC118, Lactobacillus salivarius TGB078 and Lactobacillus salivarius TGB092. All strains are indicated by their strain codes (UCC118, TGB078, TGB092). Colony forming units (CFU) per ml of culture plotted against time.

FIG. 6: Survival in the absence of thymidine of Lactobacillus salivarius UCC118, and Lactobacillus salivarius TGB092 in comparison with Lactococcus lactis MG1363 and its thyA mutant Thy12. Eighteen colonies of any of the indicated strains were inoculated in 87 ml of:
A) MRSΔT (thymidine-free MRS, enzymatically prepared by conversion of all thymidine to thymine) in the case of Lactobacillus salivarius UCC118 (wt) or Lb. salivarius TGB092 (thyA-deficient).
B) GM17ΔT (thymidine- and thymine-free GM17, prepared by bacteriological exhaustion of thymidine and thymine from GM17 by a thyA-deficient Lactococcus lactis, filtration and autoclaving and re-addition of glucose) in the case of L. lactis MG1363 (wt) or L. lactis Thy12 (thyA-deficient).

The suspensions were split and appropriate amounts of thymidine were added to one-half of either one of the suspensions to reach 1 μM.

All suspensions were aliquoted in an appropriate number of vials and these vials were incubated at 37° C. (Lactobacillus) or 30° C. (Lactococcus). Vials were opened only once to determine colony forming units (cfu) per ml, as done by triplicate plating of appropriate dilutions. In the course of this experiment, all thyA-deficient strains reached 0 cfu (i.e., zero colonies present on three plates on which 100 μl of a 1:1 dilution were plated). TGB092 reached near 0 cfu values (a maximum of one colony per plate when 100 μl of a 1:1 dilution was plated) after 24 hours and 48 hours and reached 0 cfu values after 96 hours and 72 hours in the settings with 0 μM and 1 μM thymidine, respectively.

FIG. 7: Growth after 29 hours of Lactobacillus wild-type and thyA mutants in the presence of thymine and thymidine.

The optical density at 600 nm (OD₆₀₀) of UCC118, TGB078 and TGB092 in MRS, MRS with 200 μM thymidine (MRSTd) or MRS with 800 μM thymine (MRSTm) was measured after 29 hours of growth at 37° C. OD₆₀₀ of MRS, MRSTd and MRSTm after 29 hours of growth at 37° C. was 0.000.

FIG. 8: Growth curves of two different Lactobacillus ThyA mutants in the presence of increasing concentrations thymine and thymidine.

OD₆₀₀ at 24 hours plotted against the concentration thymidine or thymine. The OD₆₀₀ at 24 hours of UCC118 when measured over the same concentration range, reached full saturation independent the thymidine or thymine concentration.

FIG. 9: Growth curves of two different Lactobacillus ThyA mutants in the presence of increasing concentrations thymine and thymidine: details at low concentration.

OD₆₀₀ at 24 hours plotted against the concentration thymidine or thymine. The OD₆₀₀ at 24 hours of UCC118 when measured over the same concentration range, reached full saturation independent the thymidine or thymine concentration.

FIG. 10: Growth of Lactobacillus salivarius UC118 at different concentrations of thymidine or thymine (OD₆₀₀ at 24 hours), showing that the lack of growth of the mutant is not due to thymine toxicity.

### DETAILED DESCRIPTION OF THE INVENTION

### Examples

### Materials and Methods to the Examples

### Media

Unless otherwise stated, Lactobacillus strains were cultivated in MRS (Merck). Special media used were:
BM9: 1 liter of 50 mM CO₃⁻ buffer at pH 8.5 supplemented with
6 g of Na₂HPO₄/3 g of KH₂PO₄1 g of NH₄Cl/0.5 g of NaCl/1 mmol of MgSO₄/0.1 mmol of CaCl₂/0.5% of glucose/0.5% of casitone (difco)

MRSΔT (MRS devoid of thymidine): MRS powder (Merck) is dissolved in an appropriate (according to the manufacturer) volume of distilled water. The solution is heated to 100° C. for one minute and allowed to cool to room temperature. 1.2 units of thymidine phosphorylase (SIGMA) are added per ml. The solution is incubated at 37° C. for 20 hours and autoclaved subsequently.

### Strains

Lactobacillus salivarius UCC118 (Dunne et al., 2001) was used as recipient strain to construct the thyA mutant.

### Example 1

### Construction of the thyA mutant

The construction of the L. salivarius thyA mutant was essentially carried out as described for Lactococcus lactis (Steidler et al., 2003), with modifications. The construction is summarized in FIG. 1. The thyA region of L. salivarius subsp. salivarius strain UCC118 was sequenced, including the upstream and downstream sequences of the coding sequence. The knowledge of these sequences is of critical importance for the genetic engineering of any Lactobacillus strain in a way as described below, as the strategy will employ double homologous recombination in the areas 1000 bp at the 5′ end and 1000 bp at the 3′ end of thyA, the “thyA target.”

In strain UCC118, the thyA gene is replaced by a synthetic gene encoding a protein that has a secretion leader, functional in Lactobacillus fused to a protein of identical amino acid sequence than the mature part of hIL-10 in which proline at position 2 had been replaced with alanine, operably linked to the Lactococcus lactis thyA promoter (PthyA, GenBank AF462070). Any combination of a promoter and the hIL-10 gene is called a hIL-10 expression cassette.

Transformation was by electroporation, at 1.5 kV, 25 mF, 400Ω, 2 mm gap length.

The thyA replacement was performed by homologous recombination, essentially as described by Biwas et al. (1993). Suitable replacements in a plasmid borne version of the thyA target are made, as described below.

The strategy involves a helper plasmid (carrying a chloramphenicol selection marker), which is brought in the target Lactobacillus strain beforehand, and a carrier plasmid (carrying an erythromycin-resistance marker), encoding the hIL-10 expression cassette flanked by upstream and downstream sequences of the chromosomal thyA gene, as described above.

The helper plasmid pTGB019 is a modified version of pVE6007. To construct pTGB019, a 3221 bp insert was generated by PCR amplification from pKD20 using the oligonucleotides GCGAAGCTTCAAATAGGGGTTCCGCGC (SEQ ID NO:17) and GCGACTAGTGGGAAAACTGTCCATACCC (SEQ ID NO:18) and cut with HindIII and SpeI. This fragment encodes the Red γ, β and exo genes under the control of the E. coli arabinose promoter and was ligated in the HindIII-SpeI opened pVE6007. This expression system, however, showed not to be functional in Lb. salivarius. The addition of arabinose to a strain carrying myc tag-labeled versions of the various RED recombinase genes did not show any expression when revealed by Western blot, neither did a Lactobacillus carrying pTGB019 show expression of either one of the RED genes as judged by intracellular protein analysis though SDS-PAGE and Coomassie brilliant blue staining. The insert will rather render the helper plasmid pTGB019 more unstable for replication in Lactobacillus when compared to pVE6007.

The carrier plasmid was electroporated into the Lactobacillus strain that holds pTGB019. Both plasmids do not stably coexist. It is at this time unclear how the mechanism of integration functions. The electroporation mixture is plated on solid agar MRS plates containing erythromycin at 10 μg/ml and thymidine at 200 μM and incubated at 42° C. for 24 hours.

The carrier plasmid is unable to replicate in Lactobacillus. Therefore, the only way to transfer the erythromycin resistance to a given strain is when a first homologous recombination, at either the 5′ 1000 bp or at the 3′ 1000 bp of the thyA target is taking place. Erythromycin-positive colonies were checked by PCR for the occurrence of such homologous recombination, as indicated in FIG. 1.

A subset of the erythromycin-resistant clones still carries pTGB019. These clones are utilized to isolate clones that show the second cross over. Appropriate dilutions were plated on MRS solid agar plates at 42° C. and from these colonies, erythroymycin- and chloramphemicol-sensitive clones were screened for the incapacity to grow in thymidine-free MRS, for the presence of both the upstream and downstream recombination, as well as for the absence of the thyA gene.

A second homologous recombination at the 3′ 1000 bp or at the 5′ 1000 bp of the thyA target yielded the desired strain. Selection for the second recombination was carried out by repeated growth in absence of erythromycin and in presence of 50 μg/ml thymidine. Colonies were tested by PCR as indicated on FIG. 1.

The resulting strains were called TGB078 (human IL-10) and TGB092 (human EL-10 operably linked to the thyA promoter).

### Example 2

### Identification of a thyA⁻ and IL-10⁺ Lactobacillus

Primary thya⁻ and IL-10⁺ Confirmation by PCR

The primary confirmation of the Lactobacillus colonies carrying a hIL-10 insert was done by PCR testing, as presented in FIG. 2. Several sets of primers were used for the detection of thyA (FIG. 2, PCR1) of IL-10 (FIG. 2, PCR2) of the flanking sequences of IL-10 (FIG. 2, PCR3 through PCR6) and of the flanking sequences of thyA (FIG. 2, PCR7 and PCR8).

The results show clearly that in the mutant strains TGB072 and TGB092, the coding sequence of thyA has been replaced by the human IL-10 sequence.

TABLE 1

primers used

| | | | | |
|---|---|---|---|---|
| | | SEQ ID | | SEQ ID |
| PCR | Forward | NO: | Reverse | NO: |

| | | | | |
|---|---|---|---|---|
| 1 | CTATAGTAGAAGAACCGTATTTAC | 1 | CAGCAACTGGCGCTTTAATTGC | 9 |
| 2 | GATTATCTCAGCTATTTTAATGTC | 2 | CGGATTTTCATAGTCATGTAAG | 10 |
| 3 | TTTAGGACAACAAAGATTGGG | 3 | GCATCACGCAAATCACGAAG | 11 |
| 4 | CTTCGTGATTTGCGTGATGC | 4 | GTCTTATTAAAGGAAGCAATTGC | 12 |
| 5 | TTTAGGACAACAAAGATTGGG | 5 | GACATTAAAATAGCTGAGATAATC | 13 |
| 6 | CTTACATGACTATGAAAATCCG | 6 | GTCTTATTAAAGGAAGCAATTGC | 14 |
| 7 | TTTAGGACAACAAAGATTGGG | 7 | GTAAATACGGTTCTTCTACTATAG | 15 |
| 8 | GCAATTAAAGCGCCAGTTGCTG | 8 | GTCTTATTAAAGGAAGCAATTGC | 16 |

Confirmation of the thyA⁻ and IL-10⁺ Properties of the Lactobacillus by Southern Blot.

To ensure that there are no thyA or IL-10 copies present elsewhere in the genome, the integration was tested by Southern blot. From the different Lactobacillus strains, a genomic DNA preparation was made. The genomic Lactobacillus DNA was digested by EcoRI and Southern blotted. The blot was revealed with digoxygenin-labeled probes for identifying thyA (thyA probe, obtained with PCR primer pair 1) or hIL-10 (hIL-10 probe, obtained with PCR primer pair 2). As expected on the basis of the PCR results, the thyA probe signal is negative and the hIL-10 probe signal on the blot is positive for the mutants, whereas the thyA probe signal is positive and the hIL-10 signal is negative for the parental strain. The results are summarized in Table 2.

TABLE 2
Expected length of PCR fragments

| Expected sizes | thyA probe | hIL-10 probe |
|---|---|---|
| UCC118 | 3757 | nihil |
| TGB078 | nihil | 3364 |
| TGB092 | nihil | 3552 |

### Example 3

### Production of Human IL-10 by the thyA⁻ and IL-10⁺ Lactobacillus

To evaluate the hIL-10 secretion, single colonies of each strain were grown in MRS supplemented with 50 μg/ml thymidine. After 40 hours of growth at 37° C., the bacteria were harvested by centrifugation and resuspended in buffered M9 (BM9) supplemented with 50 μg/ml thymidine. The suspension was incubated for five hours at 37° C., and then the prevalence of human IL-10 was determined by ELISA (Becton Dickinson). The results are summarized in FIG. 4. Both strains comprising the human IL-10 coding sequence do produce IL-10, but the production is far higher when the human IL-10 coding sequence is operably linked to the Lactococcus lactis thyA promoter. Although the production of hIL-10 is lower than what is described for Lactococcus lactis (Steidler et al., 2003), the amount is sufficiently high to be effective in vivo for the treatment of chronic intestinal inflammation.

### Example 4

### Survival in Absence of Thymidine

Survival in thymidine-free medium was tested for the two mutant strains and the parental strain. Survival was measured as colony forming units (CFU) per ml of culture, in function of the time. The results are presented in FIGS. 5 and 6.

Single colonies of all strains were inoculated in MRSΔT supplemented with 25 μg/ml of thymidine and incubated for 20 hours at 37° C. Bacteria were harvested by centrifugation, washed twice with 1V MRSΔT, resuspended in 1V of MRSΔT, diluted 1:20 in MRSΔT and incubated at 37° C. At relevant time points, CFU per ml were determined by plating on MRS solid agar plates supplemented with 50 μg/ml of thymidine.

As can be seen, the CFU is reduced by more than 2 log units after 500 minutes. A reduction of 3 log units is obtained after less than 1000 minutes. These results are far better than those obtained by Steidler et al. (2003) for Lactococcus lactis, where about twice the time is needed to obtain a reduction with 2 log units and 50 hours is needed to obtain a reduction with 3 log units.

It is important to note that these results are obtained in presence of thymine. Indeed, the thymidine is removed from the medium by enzymatic treatment, converting the thymidine in thymine. Notwithstanding the remaining concentration of thymine, the death induced by thymidine starvation is extremely fast, indicating that the strain cannot be rescued by the presence of thymine.

### Example 5

### The Lactobacillus ThyA Mutant Cannot be Rescued by Thymine

Lactobacillus salivarius UCC118 (thyA wild-type), TGB078 and TGB092 (both thyA deficient) were grown in MRS, MRS with 200 μM thymidine (MRSTd) or MRS with 800 μM thymine (MRSTm).

The optical density at 600 nm was measured after 29 hours of growth at 37° C.

The data obtained (FIG. 7) show that UCC118 reaches a comparable optical density irrespective of the growth medium. The concentration of thymidine in MRS is limiting the growth of TGB078 and TGB092. When 200 μM thymidine is added to MRS, TGB078 and TGB092 reach the same optical density as UCC118. The addition of 800 μM thymine to MRS is unable to support the growth of TGB078 and TGB092 to higher optical densities.

As can be appreciated from FIG. 7, MRS contains a substantial amount of thymidine. Thymidine can be converted to thymine with thymidine phosphorylase. MRS digested with thymidine phosphorylase thus gives MRSΔT. Lactobacillus salivarius UCC118 (thyA wild-type), TGB078 and TGB092 (both thyA deficient) were grown in MRSΔT with a range of thymidine or thymine concentrations added. After 24 hours of growth at 37° C., the cultures reach saturation. The OD₆₀₀ at 24 hours was plotted against thymidine or thymine concentration (FIGS. 8 and 9).

These results show that both thyA-deficient strains can use exogenous thymidine but not thymine for growth, whereas wild-type growth is not influenced by addition of either thymidine or thymine (FIG. 10), proving that the lack of growth is not due to thymine toxicity.

### REFERENCES

Ahmad S. I., S. H. Kirk and A. Eisenstark (1998). Thymine metabolism and thymineless death in prokaryotes and eukaryotes. Annu. Rev. Microbiol. 52:591-625.
Biswas I., A. Gruss, S. D. Ehrlich, et al. (1993). High-efficiency gene inactivation and replacement system for gram-positive bacteria. J. Bacteriol. 175:3628-3635.
Dunne C., L. O'Mahoney, L. Murphy, G. Thronton, D. Morrissey, S. O'Halloran, M. Feeney, S. Flynn, G. Fitzgerald, C. Daly, B. Kiely, G. C. O'Sullivan and F. Shanahan (2001). In vitro selection criteria for probiotic bacteria of human origin: correlation with in vivo findings. Am. J. Clin. Nutr. 73 suppl, 386S-392S.
Fu X. and J. G. Xu (2000). Development of a chromosome-plasmid balanced lethal system for Lactobacillus acidophilus with thyA gene as selective marker. Microbiol. Immunol. 44:551-556.
Goulian M., B. M. Bleile, L. M. Dickey, R. H. Grafstrom, H. A. Ingraham, S. A. Neynaber, M. S. Peterson and B. Y. Tseng (1986). Mechanism of thymineless death. Adv. Exp. Med. Biol. 195 Pt B, 89-95.
Kaplan D. L., C. Mello, T. Sano, C. Cantor and C. Smith (1999). Streptavadin-based containment system for genetically engineered microorganisms. Biomol. Eng. 31:135-140.
Knudsen S., P. Saadbye, L. H. Hansen, A. Collier, B. L. Jacobsen, J. Schlundt and O. H. Karlstrom (1995). Development and testing of improved suicide functions for biological containment of bacteria. Appl. Environ. Microbiol. 61:985-991.
Molina L., C. Ramos, M. C. Ronchel, S. Molin and J. L. Ramos (1998). Construction of an efficient biologically contained pseudomonas putida strain and its survival in outdoor assays. Appl. Environ. Microbiol. 64:2072-2078.
Pinter K., V. J. Davisson and D. V. Santi (1988). Cloning, sequencing, and expression of the Lactobacillus casei thymydilate synthase. DNA 7:235-241.
Schweder T., K. Hofmann and M. Hecker (1995). Escherichia coli K12 relA strains as safe hosts for expression of recombinant DNA. Appl. Environ. Microbiol. 42:718-723.
Steidler L., W. Hans, L. Schotte, S, Neirynck, F. Obermeier, W. Falk, W. Fier and E. Remaut (2000). Treatment of murine colitis by Lactococcus lactis secreting Interleukin-10. Science 289:1352-1355.
Steidler L., J. M. Wells, A. Raeymaekers, J. Vandekerckhove, W. Fiers, and E. Remaut (1995). Secretion of biologically active murine Interleukin-2 by Lactococcus lactis subsp. Lactis. Appl. Environ. Microbiol. 61:1627-1629.
Steidler L., S. Neirynck, N. Huyghebaert, V. Snoeck, A. Vermeire, B. Goddeeris, E. Cox, J. P. Remon and E. Remaut (2003). Biological containment of genetically modified Lactococcus lactis for intestinal delivery of human interleukin 10. Nature Biotech 21:785-789.
Tedin K., A. Witte, G. Reisinger, W. Lubitz and U. Basi (1995). Evaluation of the E. coli ribosomal rrnB P1 promoter and phage-derived lysis genes for the use in biological containment system: a concept study. J. Biotechnol. 39:137-148.
Maguin E., P. Duwat, T. Hege, D. Ehrlich and A. Gruss (1992). New thermosensitive plasmid for gram-positive bacteria. J. Bacteriol. 174:5633-5638.
Datsenko K. A. and B. L. Wanner (2000). One-step inactivation of chromosomal genes in Escherichia coli K-12 using PCR products. Proc. Natl. Acad. Sci. U.S.A. 97:6640-6645.

1. An isolated strain of Lactobacillus sp. comprising a defective recombinant thyA gene, whereby survival of said strain is strictly dependent upon the presence of thymidine.

2. The isolated strain of claim 1, wherein said Lactobacillus sp. is Lactobacillus salivarius.

3. The isolated strain of claim 2, wherein said Lactobacillus sp. is Lactobacillus salivarius subsp. salivarius strain UCC118.

4. The isolated strain of claim 1, characterized by an initial decrease in viability in absence of thymidine of at least 2 log cfu in 16 hours.

5. A method of delivering a prophylactic and/or therapeutic molecule to a subject, said method comprising:
administering to the subject the isolated strain of Lactobacillus sp. of claim 1 to the subject so as to deliver the prophylactic and/or therapeutic molecules.

6. The method according to claim 5, wherein said delivery requires biological containment under conditions wherein the thymidine and/or thymine concentration cannot be strictly controlled.

7. The method according to claim 5 wherein said prophylactic and/or therapeutic molecule is interleukin 10.

8. A pharmaceutical composition comprising:
the isolated strain of claim 1.

9. (canceled)

10. The method according to claim 5, wherein the subject has inflammatory bowel disease.

11. The isolated strain of claim 2, further characterized by an initial decrease in viability in absence of thymidine of at least 2 log colony forming units in 16 hours.

12. The isolated strain of claim 3, further characterized by an initial decrease in viability in absence of thymidine of at least 2 log colony forming units in 16 hours.

13. The method according to claim 5, wherein said Lactobacillus sp. is Lactobacillus salivarius.

14. The method according to claim 13 wherein said Lactobacillus sp. is Lactobacillus salivarius subsp. salivarius strain UCC118.

15. The method according to claim 5, characterized by an initial decrease in viability in absence of thymidine of at least 2 log colony forming units in 16 hours.

16. The method according to claim 6, characterized by an initial decrease in viability in absence of thymidine of at least 2 log colony forming units in 16 hours.

17. The method according to claim 7, characterized by an initial decrease in viability in absence of thymidine of at least 2 log colony forming units in 16 hours.

18. A method of delivering a biologically active molecule to a subject, said method comprising:
administering to the subject an isolated strain of Lactobacillus sp. comprising:
a defective recombinant thyA gene, and
a nucleic acid sequence encoding said biologically active molecule,
wherein survival of said Lactobacillus species depends upon thymidine's presence, which is characterized by an initial decrease in viability in absence of thymidine of at least 2 log colony forming units in 16 hours.

19. The method according to claim 18, wherein said method is conducted under conditions wherein the thymidine and/or thymine concentration cannot be strictly controlled.

20. The method according to claim 18 wherein said biologically active molecule is interleukin 10.

21. The method according to claim 18, wherein said Lactobacillus sp. is Lactobacillus salivarius subsp. salivarius strain UCC118.
